(19) United States
(12) Patent Application Publication
(10) Pub. No.: US 2026/0306450 A1
(43) Pub. Date: Oct. 8, 2026

(54) COMBINATION TREATMENT WITH CISPLATIN AND PG3 FOR P53-MUTATED HEAD AND NECK CANCERS

(71) Applicant: BROWN UNIVERSITY, Providence, RI (US)

(72) Inventors: Wafik El-Deiry, Providence, RI (US); Xiaobing Tian, Providence, RI (US)

(21) Appl. No.: 19/635,216

(22) Filed: Mar. 31, 2026

Related U.S. Application Data
(60) Provisional application No. 63/785,302, filed on Apr. 8, 2025.

(51) Int. Cl. A61K 31/4025; A61K 33/243; A61K 45/06

(52) U.S. Cl. CPC A61K 31/4025; A61K 33/243; A61K 45/06

(57) ABSTRACT

A method of treating cancer in a subject, comprising administering to the subject a therapeutically effective amount of a composition comprising a combination of cisplatin or a cisplatin derivative and PG3, is described. A method of preventing cisplatin resistance and cancer recurrence in a subject, comprising administering to the subject a prophylactically effective amount of a composition comprising a combination of cisplatin or a cisplatin derivative and PG3, is also described. Pharmaceutical compositions comprising cisplatin or a cisplatin derivative and PG3 are also described.

### CROSS-REFERENCE TO RELATED APPLICATIONS

This application claims priority to U.S. Provisional Application No. 63/785,302, filed Apr. 8, 2025, which is incorporated herein by reference.

### STATEMENT REGARDING FEDERALLY SPONSORED RESEARCH

This invention was made with government support under Grant Number R01 CA176289 awarded by the National Institutes of Health. The government has certain rights in the invention.

### BACKGROUND

Head and neck (H&N) cancer is a group of diverse and heterogeneous tumors that involve the oral cavity, pharynx, larynx, paranasal sinuses, nasal cavity, salivary glands, or head and neck lymph nodes, where squamous cell carcinoma (SCC) represents 90% of the diagnoses. (K. Parmar, et al., Cancer Treat and Res Commun, 33:100649 (2022)). Single treatments such as surgery or radiotherapy are typically suitable for the 30~40% of H&N cancer patients with early stage (I or II) diseases. For the approximately 60% of H&N cancer patients with locally or regionally advanced disease (III or IV), multi-modality treatment are necessary, including chemotherapy.

p53, as a transcription factor, plays its role as a tumor suppressor by activating the expression of numerous target genes. p53 encoding gene TP53 is one of the most commonly mutated genes in human tumors, with mutations detected in 65-85% of head and neck squamous cell carcinoma (HNSCC). (Li M, et al., Oncol Rep. 2023 September; 50 (3): 162).

Cisplatin, a platinum-based compound, has been used as first-line treatments for patients who have been diagnosed with various types of malignancies, including head and neck cancers. For stage III and IV HNSCC patients, cisplatin is one of standard chemotherapy drugs. DNA damage following cisplatin treatment can activate apoptosis via p53 in p53 wild-type cancer cells. In p53-mutated/deleted cancer cells, DNA-damage drugs can lead to cell death through other mechanisms, such as integrated stress response (ISR) pathway. (Tian X, et al., Front Pharmacol. 2021 Sep. 24; 12:747837). There are several mechanisms that cancer cells acquire to avoid cell death following chemotherapy, including DNA/RNA damage repair. Nucleotide excision repair (NER) remains known as the primary strategy for repair of cisplatin-induced DNA damage. Downregulation of activating transcription factor 4 (ATF4) has been reported to contribute to cisplatin resistance in gastric cancer cells. (Feng L. et al., Clin. Transl. Med. 2021; 11: e587). Elevated ERCC1 and ERCC5 expression enhance NER and are associated with cisplatin resistance in HNSCC patients. (Kanno Y, et al., Front Oncol. 2021 May 7; 11:640392).

Although many patients treated with cisplatin initially respond well, some eventually relapse and develop resistance. For treatment of locally advanced HNSCC patients, the initial cisplatin-based chemotherapy response can be up to 50%, while half of the patients remain without response to cisplatin; furthermore, most patients will develop acquired cisplatin-resistance, which induces cancer recurrence. Cisplatin-resistance and recurrence are the major factors leading to cisplatin-based therapeutic failure in HNSCC patients. Thus, it is important to develop a more suitable therapeutic approach to overcome the cisplatin-resistance and improve clinical outcome in HNSCC.

### SUMMARY OF THE INVENTION

The inventors hypothesize that (1) combination treatment of PG3 with cisplatin can reduce side effects of cisplatin through reducing the dose of cisplatin, which can be achieved by enhanced induction of integrated stress response, and (2) PG3 can sensitize cisplatin-resistant and p53-mutated H&N cancer cells to cisplatin through enhanced induction of integrated stress response.

The combination treatment shows synergistic effects in p53-mutated FaDu and Cal27 H&N cancer cells. In combination therapy, use of PG3 allows use of a lower cisplatin dose to achieve a therapeutic benefit. The combined treatment enhances ISR induction, and the ISR contributes to cell apoptosis. The inventors identified that cisplatin or the combination treatment activates the HRI/ATF4/NOXA pathway. NOXA mediates Mcl-1 degradation and is responsible for the combination treatment-induced apoptosis.

The inventors have demonstrated that small molecule PG3 can sensitize cisplatin resistant and p53 deficient (mutated/deleted/null) head and neck (H&N) cancer cells to cisplatin. The inventors have observed that the combination of PG3 and cisplatin shows synergistic effects in p53-mutated FaDu and Cal27 H&N cancer cells. Further, the combination enhances ISR induction, and the ISR contributes to cell apoptosis. The inventors also have identified that cisplatin or the combination treatment activates the HRI/ATF4/NOXA pathway, and NOXA mediates Mcl-1 degradation and is responsible for the combination treatment-induced apoptosis. The inventors have discovered that treating a subject of p53 deficient head and neck cancer cells with a combination of PG3 and cisplatin reduces the side effects of cisplatin by reducing the dose of cisplatin needed to achieve a therapeutic benefit.

### DETAILED DESCRIPTION OF THE INVENTION

The present invention provides a pharmaceutical composition comprising a combination of cisplatin or a cisplatin derivative and PG3. Methods of using the composition to treat cancer or prevent cisplatin resistance and/or cancer recurrence in a subject are also provided.

Unless otherwise defined, all technical and scientific terms used herein have the same meaning as commonly understood by one of ordinary skill in the art to which these exemplary embodiments belong. The terminology used in the description herein is for describing particular exemplary embodiments only and is not intended to be limiting of the exemplary embodiments. As used in the specification and the appended claims, the singular forms “a,” “an,” and “the” are intended to include the plural forms as well, unless the context clearly indicates otherwise. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

A “subject,” as used herein, can be any animal, and may also be referred to as the patient. Preferably the subject is a mammal, such as a research animal (e.g., a monkey, rabbit, mouse or rat) or a domesticated farm animal (e.g., cow, goat, horse, pig) or pet (e.g., dog, cat). In some embodiments, the subject is a human.

“Treat”, “treating”, and “treatment”, etc., as used herein, refer to any action providing a benefit to a subject at risk for or afflicted with a condition or disease such as cancer, including improvement in the condition through lessening or suppression of at least one symptom, delay in progression of the disease, prevention or delay in the onset of the disease, etc.

“Preventing,” as used herein, refers to any action that decreases the risk that a subject will develop cancer, inhibits the growth of cancer, or decreases the incidence of cancer recurrence. Preventing cancer can be done in subjects who have an increased risk of developing cancer. Also intended to be encompassed by this definition is the prevention of metastasis of malignant cells or to arrest or reverse the progression of malignant cells. Subjects can have an increased risk of developing cancer as a result of, for example, a genetic predisposition or exposure to carcinogens.

The terms “therapeutically effective” and “pharmacologically effective” are intended to qualify the amount of each agent that will achieve the goal of decreasing disease severity while avoiding adverse side effects such as those typically associated with alternative therapies. The therapeutically effective amount may be administered in one or more doses.

The term “prophylactically effective” is intended to qualify the amount of each agent that will achieve the goal or preventing or decreasing the risk of cancer recurrence or the development of cisplatin resistance.

All scientific and technical terms used in the present application have meanings commonly used in the art unless otherwise specified. The definitions provided herein are to facilitate understanding of certain terms used frequently herein and are not meant to limit the scope of the present application.

### Cisplatin and PG3 Compositions

In one aspect, the present invention provides a pharmaceutical composition comprising a combination of cisplatin or a cisplatin derivative and PG3 or a pharmaceutically acceptable salt thereof. Cisplatin (cis-dichlorodiamine platinum II) is a is a coordination complex of platinum having the formula cis-[Pt(NH₃)₂Cl₂]. Cisplatin derivatives are coordination complexes featuring a central platinum atom surrounded by specific ligands, maintaining a cis configuration essential for activity. See Weiss, R. and Christian, M., Drugs, 46(3):360-377(1993). Examples of cisplatin derivatives include, but are not limited to Carboplatin, Oxaliplatin, Nedaplatin, Lobaplatin, Heptaplatin, and Satraplatin.

The compound PG3 has a structure represented by the following formula:

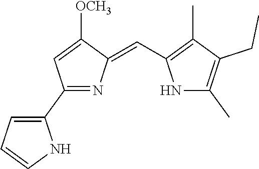 [SMILES: CCc1c(C)[nH]c(/C=C2\N=C(c3ccc[nH]3)C=C2OC)c1C]

PG3 is a highly active purified preparation of progidiosin (PG). See Islan et al., Bioengineered, 13(6):14227-14258(2022). PG3 is a derivative of PG3-Oc (U.S. Pat. No. 10,654,801) that can be obtained through removal of its ester group and shortening of its long hydrophobic side-chain while maintaining the pharmacophore of PG3-Oc, which increases solubility in water and stability in vivo. (Tian X. and El-Deiry W. S. Oncotarget. 2024; 15:614-633).

Without wishing to be bound by theory, PG3 has been shown to trigger the integrated stress response (ISR) and leads to cell apoptosis through HRI/eIF2α/ATF4/PUMA pathway in p53-mutated and deleted colorectal cancer cell lines. (Tian X. and El-Deiry W. S. Oncotarget, 15:614-633 (2024)). In many embodiments, this effect acts synergistically with the known antitumor effects of cisplatin or a cisplatin derivative.

The combination of cisplatin or a cisplatin derivative and PG3 can be brought into association with a pharmaceutically acceptable carrier or one or more other accessory ingredients, and then, if necessary or desirable, shaping or packaging the product into a desired single- or multi-dose unit. The percentages or ratio of PG3 to the cisplatin or cisplatin derivative in the combination can vary depending on the subject, the form of cisplatin, and other factors known to those skilled in the art. In some embodiments, the cisplatin or cisplatin derivative comprises about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% by weight. Alternately, the ratio of PG3 to the cisplatin or cisplatin derivative can be about 1:20, about 1:10, about 1:5, about 1:3, about 1:2, about 1:1, about 2:1, about 3:1, about 5:1, about 10:1, or about 20:1 by weight.

### Cancer Treatment Using a Combination of Cisplatin and PG3

Another aspect of the present invention provides a method of treating cancer in a subject, comprising administering to the subject a therapeutically effective amount of a pharmaceutical composition comprising a combination of cisplatin or a cisplatin derivative and PG3. The composition can comprise cisplatin or a cisplatin derivative and PG3 in any of the percentages or ratios described herein. Alternately, the cisplatin or a cisplatin derivative and PG3 can be administered to the subject concurrently, but separately. Concurrently, as referred to herein, refers to administration sufficiently proximal in time that the effects of the drug overlap. In some embodiments, the drugs are administered within an hour of each other, while in other embodiments the drugs are administered simultaneously.

Cancer cells, as defined herein, are cells that contain genetic damage that has resulted in the relatively unrestrained growth of the cells. The genetic damage present in a cancer cell is maintained as a heritable trait in subsequent generations of the cancer cell line. The cancer treated by the method of the invention may be any of the forms of cancer known to those skilled in the art or described herein. Cancer that manifests as both solid tumors and cancer that instead forms non-solid tumors as typically seen in leukemia can be treated. Based on the prevalence of an increase in aerobic glycolysis in all types of cancer, the present invention provides methods for treating a subject that is afflicted with various different types of cancers, including carcinoma, sarcoma, and lymphoma. In some embodiments, the cancer is selected from the group consisting of brain, colon, lung, pancreatic, ovarian, skin, breast, head, and neck cancers.

The effectiveness of cancer treatment may be measured by evaluating a reduction in tumor load or decrease in tumor growth in a subject in response to the administration of the combination of cisplatin and PG3 composition. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

In some embodiments, the cancer is head and neck cancer. Head and neck cancer includes a variety of different types of cancer that occur or form in the oral cavity, pharynx, larynx, paranasal sinuses, nasal cavity, salivary glands, head and neck lymph nodes, or sometimes in the salivary glands, sinuses, or muscles or nerves in the head and neck. Examples of head and neck cancers include, but not limited to mouth cancer, throat cancer, voice box cancer, paranasal sinus cancer, nasal cavity cancer, and salivary gland cancer.

In some embodiments, the cancer is squamous cell carcinoma. Squamous cell carcinomas, also known as epidermoid carcinomas, are cancers that begin in the squamous cells. Squamous cell carcinomas of the head and neck are cancer cells beginning in the squamous cells that line the mucosal surfaces of the head and neck, for example, those inside the mouth, throat, and voice box.

In some embodiments, the TP53 gene in the cancer is mutated. TP53 gene mutations refer to alterations in the TP53 DNA/RNA sequence. The mutation can be a missense mutation due to various nucleotide substitutions, insertions, or deletions, creating a substitution of an amino acid in the p53 protein. TP53 mutation can be a nonsense mutation, creating a premature termination codon that leads to the expression of truncated inactive p53 protein. TP53 mutation can also be a frameshift mutation altering the basic integrity of p53 protein. TP53 mutations can be disruptive mutations leading to disruption of p53 protein, and non-disruptive mutations that retain some of functional properties of wild type p53 protein. In some embodiments, the TP53 gene in the cancer is absent or deleted.

In some embodiments, p53 protein in the cancer is mutated. p53 protein is the translated product of TP53 gene. P53 protein mutations have an altered function, such as an impaired p53 function, or a gain of function of p53. In some embodiments, p53 protein in the cancer is deleted or absent.

In some embodiments, the cancer is cisplatin resistant. In some examples, cisplatin resistant cancer can be an intrinsic cisplatin resistant cancer, where innate resistance exists prior to administering with or exposure to cisplatin, resulting in reduced efficacy of cisplatin treatment. Examples of intrinsic cisplatin resistant cancers include, but not limited to cancers caused by (1) pre-existing (inherent) genetic mutations that result in decreased responsiveness; (2) heterogeneity of tumors in which pre-existed insensitive subpopulations, including cancer stem cells, will be selected thus leading to relapse in later stages of therapeutic treatment; or (3) activation of intrinsic pathways used as defense against environmental toxin including anti-cancer drugs.

In other examples, cisplatin resistant cancer can be an acquired cisplatin resistant cancer. Acquired cisplatin resistance can be a resistance develops after administering with or exposure to cisplatin, and the cisplatin efficacy reduces after initial response. Generally, an acquired anti-cancer agent resistance can be a result of (1) activation of second proto-oncogene that becomes the newly emerged driver gene; (2) mutations or altered expression levels of the drug targets; (3) changes in tumor microenvironment (TME) after treatment. Examples of acquired cisplatin resistant cancers include, but not limited to cancers caused by changes (increase or decrease) of cisplatin import or export, increased cisplatin inactivation, increased cisplatin detoxification, increased DNA damage repair, and inactivated cell death signaling. In other embodiments, the cancer is cisplatin sensitive.

In some examples, the cisplatin resistance cancer is contributed by tumor suppressor gene TP53 and/or its encoded transcriptional factor p53 protein. Activation of p53 and its downstream pathways (e.g. cell-cycle arrest, apoptosis, senescence, or other antiproliferative programs) helps cisplatin to exert its antitumor effects. p53 mutations can lead to the inactivation of cisplatin targets, leading to cisplatin resistance. Additionally, some p53 mutations can show a gain of function effect that further leads to cisplatin resistance. Chemotherapy with cisplatin carries the risk of causing mutations in TP53 gene. If p53 mutations accumulate during cisplatin chemotherapy, efficacy of cisplatin therapy may be further affected. In other examples, the cisplatin resistance cancer is independent of p53 status. For example, integrated stress response (ISR) pathway, and MCL-1/ATF4/NOXA pathway mediated cell death have been implicated in cisplatin resistance in an p53 independent manner.

Methods of cancer treatment using a composition comprising a combination of cisplatin or a cisplatin derivative and PG3 can further include the step of ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, and administration of immunotoxins. The use of an additional method of cancer treatment can be used before, during, or after treatment with the combination of cisplatin and PG3. In some embodiments, the method further comprises treatment of the cancer using immunotherapy, chemotherapy, phototherapy, or radiotherapy.

Methods of treating cancer using a composition comprising a combination of cisplatin or a cisplatin derivative and PG3 include simultaneous administration, or administration of cisplatin or the cisplatin derivative close in time to the administration of PG3.

The above methods of treating cancer may also include a step of identifying a subject that is likely to have cisplatin resistance. The subject can be identified by determining the level of a cisplatin response biomarker known in the art from a biological sample, e.g., TP53 gene, p53 protein, survivin, ERCC-1, VRAC, etc. Biological samples include, but are not limited to a cell sample, or a tissue sample, or a tumor sample. Another example of a biological sample is bodily fluids such as saliva, urine and blood-related samples (e.g., whole blood, serum, plasma, peripheral blood lymphocytes (PBMC) and other blood-derived samples), urine, cerebral spinal fluid, bronchoalveolar lavage, and the like. The levels of an indicator in a biological sample taken from the subject can be assessed either quantitatively or qualitatively, and can be determined either in vivo or in vitro. In some embodiments, the biological sample comprises blood, plasma, or serum.

The biological sample can be provided by immediately obtaining a biological sample from the subject, or a biological sample can be provided that was previously obtained. A biological sample may be fresh or stored (e.g., blood or blood fraction stored in a blood bank). Samples can be stored for varying amounts of time, such as being stored for an hour, a day, a week, a month, more than a month, years and decades of storage. The biological sample may be a bodily fluid expressly obtained for use in the methods of this invention or can be a bodily fluid obtained for another purpose which can be subsampled for the assays of this invention.

In some embodiments, the indicator is a polynucleotide, while in other embodiments, the indicator is a polypeptide. The level of indicator in a subject can be measured using an analytic device. The analytic device used to measure the levels of indicator can be either a portable or a stationary device. In addition to including equipment used for detecting indicators, the analytic device can also include additional equipment to provide purification (i.e., physical separation) of analytes prior to analysis.

Polypeptide indicators may be determined by any of a variety of standard or novel recently developed protein analytic methods known in the art. These methods include absorbance, gel electrophoresis (e.g., SDS-PAGE gel purification), a protein immunoblot (e.g., western blot), chromatography (e.g., size exclusion chromatography, ion exchange chromatography, and affinity chromatography), precipitation, ultracentrifugation, an immunoassay, such as an enzyme-linked immunosorbent assays (ELISA), and other common techniques known to one of ordinary skill in the art. Novel recently developed assays include bead based immunofluorescence assays that can quantitatively measure different proteins in the same well with high sensitivity.

Polynucleotide indicators may be determined by any of a variety of standard analytic methods known in the art. In one embodiment of the invention, the expression level of an indicator gene (which is a polynucleotide) is determined by nucleic acid amplification of said gene using gene specific primer pairs, and quantifying the amplification results. In some embodiments, the nucleic acid amplification method used is real-time polymerase chain reaction (PCR) analysis.

### Cisplatin Resistance and Cancer Recurrence Prevention Using a Combination of Cisplatin and PG3

Another aspect of the present invention provides a method of preventing cisplatin resistance and/or cancer recurrence in a subject, comprising administering to the subject a prophylactically effective amount of a composition comprising a combination of cisplatin or a cisplatin derivative and PG3. Methods of preventing cisplatin resistance and/or cancer recurrence can also be used to decrease the risk that a subject will develop a recurrence of cancer or cisplatin resistance. The method can be used is a subject identified as having an increased risk of developing cisplatin resistance or cancer recurrence. Factors relating to the likelihood of cancer recurrence include the type of cancer, its stage, and the medical history of the subject. The risk of cisplatin resistance is frequently linked to more aggressive and metastatic cancer, although genetic and epigenetic factor also play a role. Lugones et al., Biomolecules, 12 (10): 1365 (2022). of preventing cisplatin resistance and cancer recurrence using a pharmaceutical composition comprising a combination of cisplatin or a cisplatin derivative and PG3 can be conducted before, during, and after cisplatin or PG3 have been employed one.

In some embodiments, the cancer is head and neck cancer. In other embodiments, the cancer is squamous cell carcinoma. In some embodiments, the TP53 gene in the cancer is mutated. In other embodiments, the TP53 gene in the cancer is absent or deleted. In some embodiments, p53 protein in the cancer is mutated. In other embodiments, p53 protein in the cancer is absent or deleted. In some embodiments, the recurrence of cancer will be prevented or its risk decreased. On other embodiments, the development of cisplatin resistance will be prevented or its risk decreased.

### Formulations and Methods of Administration

“Delivery vehicle” refers to any compositions that acts to assist, promote or facilitate entry of the provided therapeutic agent complexes to biological systems to achieve a therapeutic effect in a subject. Such delivery vehicles, also referred to as pharmaceutically acceptable carriers, including any of the formulations described herein, or known to those skilled in the art as suitable for use in administering the active agents described herein.

The formulations of the pharmaceutical compositions described herein may be prepared by any method known or hereafter developed in the art of pharmacology. In general, such preparatory methods include the step of bringing the active ingredient into association with a pharmaceutically acceptable carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredients, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) of either active ingredient (i.e, PG3 and Cisplatin or a Cisplatin Derivative).

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredients combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredients, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The active compounds described herein can also be provided as pharmaceutically acceptable salts. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of the compounds may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, γ-hydroxybutyric, galactaric, and galacturonic acids. Suitable pharmaceutically acceptable base addition salts of the compounds described herein include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine) and procaine may be used form base addition salts of the compounds described herein. All of these salts may be prepared by conventional means from the corresponding compounds described herein by reacting, for example, the appropriate acid or base with the compound.

The complete disclosure of all patents, patent applications, and publications, and electronically available material cited herein are incorporated by reference. The foregoing detailed description and examples have been given for clarity of understanding only. No unnecessary limitations are to be understood there from. The invention is not limited to the exact details shown and described, for variations obvious to one skilled in the art will be included within the invention defined by the claims.

What is claimed is:

1. A method of treating cancer in a subject, comprising administering to the subject a therapeutically effective amount of a pharmaceutical composition comprising a combination of cisplatin or a cisplatin derivative and PG3 or a pharmaceutically acceptable salt thereof.

2. The method of claim 1, wherein the pharmaceutical composition further comprises a delivery vehicle.

3. The method of claim 1, wherein the cancer is squamous cell carcinoma.

4. The method of claim 1, wherein the cancer is head and neck cancer.

5. The method of claim 1, wherein the cancer is mouth cancer.

6. The method of claim 1, wherein the cancer is throat cancer.

7. The method of claim 1, wherein TP53 gene or p53 protein in the cancer is mutated or deleted.

8. The method of claim 1, wherein the cancer is cisplatin sensitive.

9. The method of claim 1, wherein the cancer is cisplatin resistant.

10. The method of claim 1, wherein the subject is a human subject.

11. The method of claim 1, wherein the method further comprises treatment of the cancer using immunotherapy, chemotherapy, phototherapy, or radiotherapy.

12. A method of preventing cisplatin resistance and cancer recurrence in a subject, comprising administering to the subject a prophylactically effective amount of a pharmaceutical composition comprising a combination of cisplatin or a cisplatin derivative and PG3.

13. The method of claim 12, the composition further comprises a delivery vehicle.

14. The method of claim 12, wherein the cancer is head and neck cancer.

15. The method of claim 12, wherein the cancer is squamous cell carcinoma.

16. The method of claim 12, wherein the cancer is mouth cancer.

17. The method of claim 12, wherein the cancer is throat cancer.

18. The method of claim 12, wherein TP53 gene or p53 protein in the cancer is mutated or deleted.

19. The method of claim 12, wherein the subject is a human subject.

20. The method of claim 12, wherein the method further comprises immunotherapy, chemotherapy, phototherapy, or radiotherapy.
